(19) United States
(12) Patent Application Publication
(10) Pub. No.: US 2013/0195802 A1
(43) Pub. Date: Aug. 1, 2013

(54) Cellular Constituents From Bacteroides, Compositions Thereof, and Therapeutic Methods Employing Bacteroides or Cellular Constituents Thereof

(71) Applicant: Brenda E. Moore, Springfield, OH (US)

(72) Inventor: Brenda E. Moore, Springfield, OH (US)

(73) Assignee: MOORE RESEARCH ENTERPRISES LLC, Srpringfield, OH (US)

(21) Appl. No.: 13/701,350

(22) PCT Filed: Jun. 1, 2011
(86) PCT No.: PCT/US11/38742

§ 371 Date: Apr. 11, 2013

Related U.S. Application Data
(60) Provisional application No. 61/350,193, filed on Jun. 1, 2010.

(51) Int. Cl. A61K 35/74; A23L 1/30

(52) U.S. Cl. CPC A61K 35/74; A23L 1/30
USPC 424/93.2; 424/780; 536/23.1; 536/123.1; 536/1.11; 530/350; 530/395; 530/359; 424/93.4; 435/252.3

(57) ABSTRACT

A cellular constituent is lysed from, produced by and/or isolated from one or more bacteria from the genus Bacteroides, and the cellular constituent, a derivative thereof, and/or one or more bacteria from the genus Bacteroides, or a modified form thereof, is employed in compositions and methods for modulating an inflammatory response. Such methods include methods of treating, delaying the onset of or reducing the symptoms of one or more inflammatory conditions/diseases, including corporal or gastrointestinal inflammation, for example, Irritable Bowel Syndrome, Crohn's Disease, or colitis, and/or associated diseases such diabetes, asthma, multiple sclerosis, cancer, rheumatoid arthritis, gingivitis, atopic diseases, for example, hay fever, food allergies, eczema, rhinitis, dermatitis, conjunctivitis, atopic syndrome and keratosis pelaris, ocular inflammatory disease, strokes, cardiovascular disease, depression, atherosclerosis and hypertension, and comprise administering a composition comprising one or more natural and/or modified bacteria of the genus Bacteroides, and/or a cellular constituent lysed from, produced by, or isolated from one or more natural and/or modified bacteria from the genus Bacteroides, or a derivative thereof.

### FIELD OF THE INVENTION

The present invention is directed to cellular constituents from a bacteria from the genus Bacteroides, and derivatives thereof, compositions including such a cellular constituent or derivative thereof, and methods employing such a cellular constituent or derivative thereof, or a bacteria from the genus Bacteroides, or a genetically modified form thereof, including methods for treating, delaying the onset of, or reducing the symptoms of one or more inflammatory conditions/diseases, including corporal or gastrointestinal inflammation, and/or associated diseases such as diabetes, asthma, multiple sclerosis, cancer, rheumatoid arthritis, gingivitis, atopic diseases, ocular inflammatory disease, strokes, cardiovascular disease, depression, atherosclerosis, and hypertension.

### BACKGROUND OF THE INVENTION

Health of the human host is reliant upon the immune system's ability to recognize and adapt to countless foreign and self molecules and respond in appropriate ways, thereby assuring maintenance of host homeostasis. Several recent studies indicate that gastrointestinal microbiota play a key role in proper function of the immune system as well as in the prevention of the initiation of the inflammatory response, with its subsequent negative impact on disease states such as inflammatory bowel disease, Type 2 Diabetes (TD2) and cardiovascular disease (CVD). Abdominal obesity, with its subsequent increase in fat deposition within omental adipocytes, has been associated with the inflammatory process as well as an increased risk of developing many diseases. It is commonly understood that the omentum is the most prolific endocrine organ within the human host. As the adipocyte mass increases, secretory products such as cytokines, for example interleukins 1 and 6, and Tumor Necrosis Factor alpha (TNF-alpha) also increase, while adiponectin (a molecular insulin sensitizer) is decreased (Kojima, S., et al., 2005. Levels of the adipocyte-derived plasma protein, adiponectin, have a close relationship with atheroma. Thromb. Res. 115:483; Ryo, M. et al., 2004. Adiponectin as a biomarker of the metabolic syndrome. Circ. J. 68:975). This, in turn, distorts hepatic metabolism, causing a surge in blood lipids, and promotes proliferation of the vasa vasorum within the arterial media, migration of macrophages and subsequent damage to the circulatory system through inflammatory processes, increasing arterial damage (Corti, R., et al., 2004. Evolving concepts in the triad of atherosclerosis, inflammation and thrombosis. J. Thromb. Thromolysis. 17:35). Evidence suggests that the gut microbiota play a role in the inflammatory process through the invocation of the cytokine/chemokine response of the immune system and may significantly affect the initiation and promotion of many physiological processes involved in development of disease. Modulation of the bacterial system may help ameliorate or decrease the symptoms/severity of such inflammation-associated disease processes including CVD, diabetes, inflammatory bowel diseases, hypertension, asthma, multiple sclerosis and cancer, etc. (Skurk, T. and H. Hauner. 2004. Obesity and impaired fibrinolysis: role of adipose production of plasminogen activator-1. Int. J. Relat. Metab. Disord. 28:1357; Corti, R., et al., 2004. Evolving concepts in the triad of atherosclerosis, inflammation and thrombosis. J. Thromb. Thromolysis. 17:35). Additionally, it has recently been suggested that the genome of the microbial population is critical in maintenance of host health overall and that the total host genome is insufficient in and of itself to support all functions necessary to support host homeostasis (Zaneveld, J., et. al., 2008. Host-bacterial co-evolution and the search for new drug targets. Curr. Opin. Chem. Biol. 12:109).

Type 2 diabetes (T2D) is commonly associated with obesity and metabolic syndrome (Hu, F. B., et. al., 2001. Diet, Lifestyle and the risk of type 2 diabetes mellitus in women. New Engl. J. Med. 345:790; Alberti, K. G. and P. Z. Zimmei. 1998. Definition, diagnosis and classification of diabetes mellitus and its complications, part 1: diagnosis and classification of diabetes mellitus, provisional report of a WHO consultation. Diab. Med. 15:539). Although the exact mechanisms have not been completely elucidated, it is common knowledge that chronic, low-grade obesity-induced inflammatory responses, for example through the activation of protein kinases such as IkB kinases (IKK) and Jun kinases (JNKs) are important factors (Hotamisligil, G. S. 2006. Inflammation and metabolic disorders. Nature. 444:860; Shoelson, S. E., et al., 2006. Inflammation and insulin resistance. J. Clin. Invest. 116:1793; White, C. R. 2003. Insulin signaling in health and disease. Science.302:1710; Solinas, G. C., et. al., 2007. JNK1 in hematopoieticlly derived cells contributes to diet-induced inflammation and insulin resistance without affecting obesity. Cell Metabol. 6:386). Pathogen-associated molecular patterns (PAMPs) from varying genera and species of bacteria are known to elicit the IKK inflammatory response. (Doyle, S. L. and L. A. O'Neill. 2006. Toll-like receptors: from the discovery of NF-kB to new insights into transcriptional regulations in innate immunity. Biochem. Pharmacol. 72(9):1102).

Irritable Bowel Syndrome (IBD) is associated with a shift from regulated intestinal immune response to one typified by unrestrained immunological cellular activity and pro-inflammatory cytokine production (De Winter, H., et al., 1999. Mucosal immunity and inflammation. II. The yin and yang of T cells in intestinal inflammation: pathogenic and protective roles in a mouse colitis model. Am. J. Physiol. 276:G1317; Simpson, S. J., et al., 2000. Pathways of T cell pathology in models of chronic intestinal inflammation. Int. Rev. Immunol. 19:1; Elson, C. O., et al., 2007. Monoclonal anti-interleukin 23 reverses active colitis in a T cell-mediated model in mice. Gastroenterology 132:2359). IBD encompasses Crohn's Disease and ulcerative colitis, both of which have been associated with GI microbiota (Podolsky, D. K., 2002. The current future understanding of inflammatory bowel disease. Best Pract. Res. Clin. Gastroenterol. 16:933; Shanahan, F. 2002. Crohn's Disease. Lancet. 359:62-69; Targan, S. R. and L. C. Karp. 2005. Defects in mucosal immunity leading to ulcerative colitis. Immunol. Rev. 206:296). Experimental evidence also indicates that transfer of populations of colitogenic microorganisms to wild-type mice was sufficient to induce experimental ulcerative colitis (Garrett, W. S., et al., 2007. Communicable ulcerative colitis induced by T-bet deficiency in the innate immune system. Cell. 131:33), demonstrating the role of bacteria in this disease process. In humans, shifts in GI bacterial populations have also been associated with IBD (Lepage, P. et al., 2005. Biodiversity of the mucosa-associated microbiota is stable along the distal digestive tract in healthy individuals and patients with IBD. Inflamm. Bowel Dis. 11:473; Scanlan, et al., 2006. Culture-independent analyses of temporal variation of the dominant fecal microbiota and targeted bacterial subgroups in Crohn's disease. J. Clin. Microbiol. 40:3980; Frank, D. N. et al., 2007. Molecular-phylogenetic characterization of microbial community imbalances in human inflammatory bowel diseases. Proc. Natl. Acad. Sci. USA. 104:13780).

Investigations into such diseases as asthma indicate that those persons afflicted with this disorder have lower populations of GI Bacteroides than the normal, non-asthmatic population (Bjorksten, B. 1999. The environmental influence on childhood asthma. Allergy. 54:517). Additional evidence from epidemiological studies have indicated that there is a link between altered GI microbiota and atopic eczema and rheumatoid arthritis (Penders, J. et al., 2007. Gut microbiota composition and development of atopic manifestations in infancy: the KOALA Birth Cohort Study. Gut. 56:661; Kalliomaki, M. and E. Isolauri. 2002. Pandemic of atopic diseases—a lack of microbial exposure in early infancy? Curr. Drug Targets Infect. Disord. 2:193; Kalliomaki, M. and E. Isolauri. 2003. Role of the intestinal flora in the development of allergy. Curr. Opin. Allergy Clin. Immunol. 3:15). In addition, several epidemiological and clinical reports have disclosed an increased incidence of immune-associated disorders, such as IBD, asthma, diabetes, rheumatoid arthritis, multiple sclerosis, and cancer (Luptin, J. R., 2004. Microbial degradation products influence colon cancer risk: the butyrate controversy. J. Nutr.134:479; Bjorksten, B. 1999. The environmental influence on childhood asthma. Allergy. 54:517; Frank, D. N., et. al., 2007. Molecular-phylogenetic characterization of microbial community imbalances in human inflammatory bowel diseases. Proc. Natl. Acad. Sci. USA. 104:13780), the rapidity of which cannot be contributed solely to increases in genetic predisposition (Noverr, M. C. and G. B. Huffnagie. 2004. Does the microbiota regulate immune responses outside the gut? Trends Microbial. 12:562).

### SUMMARY OF THE INVENTION

The present invention is directed to cellular constituents, compositions containing such constituents or a derivative thereof, and methods for modulating an inflammatory response.

More specifically, in one embodiment, the invention is directed to a cellular constituent lysed from, produced by, or isolated from one or more species of bacteria from the genus Bacteroides, or a derivative thereof. In another embodiment, the invention is directed to compositions comprising such a cellular constituent or derivative thereof.

In another embodiment, the invention is directed to a genetically modified form of bacteria from the genus Bacteroides. In another embodiment, the invention is directed to compositions including a genetically modified form of bacteria from the genus Bacteroides.

In another embodiment, the invention is directed to methods for treating, delaying the onset of (including reducing the risk of developing), or reducing the symptoms of corporal or gastrointestinal inflammation in an individual, and, more specifically, to methods for treating, delaying the onset of, or reducing the symptoms of one or more inflammatory conditions/diseases, including corporal or gastrointestinal inflammation, for example, Irritable Bowel Syndrome, Crohn's Disease, or colitis, and/or associated diseases such diabetes, asthma, multiple sclerosis, cancer, rheumatoid arthritis, gingivitis, atopic diseases, for example, hay fever, food allergies, eczema, rhinitis, dermatitis, conjunctivitis, atopic syndrome and keratosis pelaris, ocular inflammatory disease, strokes, cardiovascular disease, depression, atherosclerosis and hypertension. The methods comprise administering a composition comprising one or more species of from the genus Bacteroides, a genetically modified form of bacteria from the genus Bacteroides, or a cellular constituent lysed from, produced by, or isolated from a bacteria from the genus Bacteroides, or a derivative thereof.

Additional embodiments of the invention will be apparent from the following detailed description.

### DETAILED DESCRIPTION

There are two main classifications of bacteria found within the human gastrointestinal tract: Gram-positive bacteria and Gram-negative bacteria, defined primarily by differences within the bacterial cell wall components. Lipopolysaccharides (LPS) are integral components of Gram-negative bacterial cell walls while techoic acids (TA), lipotechoic acid (LA) and peptidoglycans (PD) are associated with the cell walls of Gram-positive bacteria. These various components are recognized by human epithelial cells to which bacterial cells can adhere by means of adhesins or ligands and elicit cellular response. In addition to intact bacterial cells, the various cell wall components (LPS, TA, LA and PD), which form pathogen-associated molecular patterns (PAMPs), also called microbial-associated molecular patterns (MAMPs), can interact with host cells. These components are released during growth or when bacteria are engulfed by host defense cells or lysed by antibiotics. Toll-like receptors (TLRs) are pattern recognition receptors (PRRs) which are linked to the innate immune response through NF-kB. Entire intact bacterial cells or the MAMPs alone can then bind to PRRs, such as the TLRs, on the host epithelial cell to elicit specific cellular responses (Muta, T and K. Takeshige. 2001. Essential roles of CD14 and lipopolysaccharide-binding protein for activation by distinguished ligands in LPS preparations. Eur. J. Biochem. 268(16):4580). The entire bacterium can also be engulfed by a GI dendritic cell which then migrates to mesenteric lymph nodes, where they induce naïve B cells to produce IgA (Macpherson, A. J. and T. Urh. 2004. Induction of protective IgA by intestinal dendritic cells carrying commensal bacteria. Science: 303:1662). It has been proposed that the secretion of IgA by GI cells may be the means by which GI microbes influence the host immune system (Cerutti, A. 2008. The regulation of IgA production class switching. Nature Rev. Immunol. 8:421; Tezuka, et al., 2007. Regulation of IgA production by naturally occurring TNF/iNOS-producing dendritic cells. Nature 448:929). Alternatively, MAMP molecules, acting in an antigen (Ag) capacity, can be transported across the epithelial cells of the gastrointestinal (GI) tract, where they are picked up by binding proteins and carried in the serum. They are then delivered to immune cells, for example, that have TLRs, which have been identified as PRR specific for PAMPs (Doyle, S. L. and L. A. O'Neill. 2006. Toll-like receptors; from the discovery of NF-kB to new insights into transcriptional regulations in innate immunity. Biochem. Pharmacol. 72(9):1102), where they bind and initiate phosphorylation of Inhibitory Kappa B kinase 2 (IKK). Once bound, nuclear factor-kappa beta (NF-kB) is activated.

Nuclear factor-kappa beta is a family of rapid acting transcription factors, i.e. transcription factors present in cells in the inactive state and which do not require new protein synthesis for activation. Thus, activation of TLR receptors results in fairly rapid changes in genetic expression. In the inactive form, NF-kB is found in the cytoplasm and bound to the inhibitory protein IkBa. Once the TLR receptors are activated by the PAMPs, the enzyme IkB kinase (IKK) is activated, phosphorylating the inhibitory protein and releasing NF-kB in the activated state. It is then translocated into the nucleus, where it binds to response elements (RE), recruiting other proteins and ultimately activating RNA polymerase. This results in transcription of DNA to mRNA, which is translated into proteins in the cytoplasm, and which then alter cell function (Brasier, A. R. 2006. The NF-kB regulatory network. Cardiovasc. Toxicol. 6(2): 111; Gilmore, T. D. 1999. The Rel/NF-kB signal transduction pathway: introduction. Oncogene 18(49):6842). Overall, activation of specific genes by NF-kB result in specific cellular/physiologic responses, for example an inflammatory or immune response (Nelson, D. E. et al, 2004. Oscillations in NF-kB signaling control the dynamics of gene expression. Science:306(5696):704). Altering the bacterial populations of the gut or presenting different molecular constituents (MAMPs) to the GI epithelial cells may positively alter genetic expression and the subsequent harmful immunologic response, thus modulating or preventing the inflammatory state and its related diseases. For example, prevention of the initiation of inflammation by blocking and/or altering the TLR receptor response (including subsequent release of cytokines) and thus preventing the NF-kB inflammatory cascade is beneficial in preventing cellular proliferation and supporting apoptosis (Lin, W -W and M. Karin, 2007. A cytokine-mediated link between innate immunity, inflammation, and cancer. J. Clin. Invest. 117(5):1175-1183), and eliminating and/or minimizing inflammation-associated disease processes in the host.

In general, the immune system consists of two different components, innate immunity and adaptive immunity. These two systems collaborate to protect the host from invasive pathogens. The innate immune system is generalized and recognizes molecular patterns such as MAMPs and encompasses general molecular components and cellular mechanisms such as TLRs, monocytes and neutrophils. Designed to prevent infection, it includes the skin/epithelial cells, and mucus secretions, which provide the first barriers in preventing adhesion and invasion by pathogenic organisms. Fast-acting, the innate system is invoked quickly and can eliminate threats to the host within hours of exposure, preventing the inflammatory response. Should this system fail, adaptive immunity then responds to eliminate the invading organism.

Anatomy of the human host defense systems are designed to protect against microbial pathogenic invaders. These mechanisms include physical barriers (epithelia in skin, respiratory, urogenital and gastrointestinal layers) and cell surface receptors (CSR) which recognize pathogens vs “self” and, when recognized, elicit specific cellular/genetic response. In general, adhesion of bacterial cells to host cell surfaces is not only needed to elicit infection but also to establish a normal gastrointestinal flora. Adhesins are molecules which mediate adhesion and are typically found on bacterial cell surfaces or on the tips of bacterial fimbriae or pili (Hultgren, S. J, et. al., 1993. Pilus and nonpilus bacterial adhesins:assembly and function in cell adhesion. Cell. 73:887).

The entire bacterial cell may not be required to initiate or prevent initiation of the host immunological defense system. Molecules derived from specific bacteria have been shown to promote immunological function within the host. The single molecule polysaccharide A (PSA), derived from Bacteroides fragilis, demonstrated the ability to direct the development of the immune system in germ-free mice (Mazmanian, S. K., et al., 2005. An immunomodulatory molecule of symbiotic bacteria directs maturation of the host immune system. Cell. 122:107). Either colonization of germ-free mice with B. fragilis or treatment with purified PSA can protect against induction of experimental IBD and decrease secretions of pro-inflammatory cytokines such as TNF, IL-17 and IL-23, associated with disease in these models (Mazmanian, S. K., et al., 2008. A microbial symbiosis factor prevents intestinal inflammatory disease. Nature. 453:620). Additionally, colonization of germ-free mice with Bacteroides thetaiotomicron suggested that this bacterium produces no inflammatory response (Hooper, L. V., et al., 2001. Molecular analysis of commensal host-microbial relationships in the intestine. Science 291:881), which in turn may decrease or prevent disease processes associated with and exacerbated by chronic inflammation.

Matrix metalloproteinases (MMP) are a family of enzymes involved in several different physiologic processes, including embryonic development, tissue remodeling, apoptosis, arthritis and host immunity. Matrylisin (MMP-7) is known to function in both tissue repair and mucosal defense (Bals, R., et al., 1998. Mouse beta-defensin 1 is a salt-sensitive antimicrobial peptide present in epithelia of the lung and urigenital tract. Infect. Immun. 66:1225). Several studies indicate that this enzyme also functions in the degradation and processing of several other matrix proteins, including elastin, proteoglycan, core proteins and serpins (Murphy, G., et al., 1991. Matrix metaloproteinase degradation of elastin, type IV collegan and proteoglycan. A quantitative comparison of the activities of 95 kDa and 78 kDa gelatinases., stromylesins-1 and −2 and punctuated metalloproteinases (PUMP). Biochem. J. 277:277; Sires, I., G., et al., 1993. Degradation of entactin by matrix metaloproteinases. Susceptibility to matrylisin and identification of cleavage sites. J. Biol. Chem. 268:2069; Halpert, L., et al., 1996. Matrilysin is expressed by lipid-laden macrophages at sites of potential rupture in atherosclerotic lesions and localizes to areas of versican deposition, a proteolytic substrate for the enzyme. Proc. Natl. Acad. Sci. USA. 93:9748).

Unlike many enzymes within the MMP family, matrylisin is expressed by non-injured exocrine and mucosal cells, particularly those with heavy bacterial loads (Wilson, C. L., et al., 1999. Regulation of intestinal alpha-defensin activation by the metalloproteinase matrilysin in innate host defense. Science 286:113; Saarialho-Kere, U. K., et al., 1993. Divergent mechanisms regulate interstitial collagenase and 92 kDa gelatinase expression in human monocyte-like cells exposed to bacterial endotoxin. J. Biol, Chem. 268:17354). Although matrilysin does not have a bacteriocidal effect, it appears to be necessary for activation of cryptins (enteric alpha-defensins) that have broad antimicrobial activity (Ouettlette, A. J., et al., 1994. Mouse Paneth cell defensin: primary structures and antibacterial activities of numerous cryptdin isoforms. Infect. Immun. 62:5040; Ouellette, A. J. and S. E. Selsted. 1996. Paneth cell defensins: endogenous peptide components of intestinal cell defense. FASEB (Fed. Am. Soc. Exp. Biol. J.). 10:1280), and thus plays a significant role in innate host defense at mucosal surfaces. Colonization of germ-free mice with a culture of Bacteroides thetaiotomicron induced matrilysin expression by Paneth cells, indicating that host immunologic defense against pathogens at the GI cell wall is enhanced by exposure to this bacterium. Evidence also suggests that the intact bacterium is not necessary to invoke a positive host immunologic response. When human colonic cell cultures (HT29) were exposed to bacterial broth filtrates, matrilysin expression occurred even when broths were treated with cyloheximide and/or antibiotics (Lopez-Baodo, Y. S., et al., 2000. Bacterial exposure induces and activates matrilysin in mucosal epithelial cells. J .Cell Biol. 148:1305). Earlier evidence also indicated that soluble bacterial factors, or modulins, stimulate immunologic/cytokine responses (Henderson, B., et. al., 1998. Bacterial modulins: a novel class of virulence factors which cause host tissue pathology by inducing cytokine synthesis. Microbiol. Rev. 60:316; Wilson, M. R. Seymour and B. Henderson. 1998. Bacterial perturbation of cytokine networks. Infect. Immun. 66:2401). These data suggest that a bacterial soluble factor is present. Such molecules from a Bacteroides species could be utilized in future applications to modulate the host inflammatory/disease response. Cellular constituents isolated or synthesized from any species within the Bacteroides genus may be isolated and utilized to modulate the inflammatory response and thus decrease the effect or prevent the onset of inflammation and associated diseases.

The utilization of germ-free (gnotobiotic) animals in studies designed to elucidate the role of microorganisms upon development of the host immune system have produced several insights. For example, germ-free mice show impairment in the development and maturation of isolated lymphoid follicles which is corrected upon introduction of gut bacteria normally found in the host's GIT (Hultgren, S. J, et al., 1993. Pilus and nonpilus bacterial adhesins:assembly and function in cell adhesion. Cell 73:887). In addition, germ-free mice have demonstrated a decrease in secretory immunoglobulin A (IgA) in the intestine (Peterson, D. A., et al., 2007. IgA response to symbiotic bacteria as a mediator of gut homeostasis. Cell Host Microbe 2:328), the functions of which include coating pathogenic bacteria to prevent adherence to host GI epithelial cells and/or binding of antigenic bacteria together to facilitate elimination, thereby preventing invasion of pathogenic organisms and thus infection, therefore precluding the initiation of the inflammatory response. While it remains unclear as to what the specific role is, evidence is now emerging to support the idea that symbiotic bacteria are actively involved in the protective secretion of IgA. IgA production is induced from naïve B cells when dendritic cells, carrying commensal bacteria or MAMPs, migrate to mesenteric lymph nodes where naïve B cells are located (Suzuki, K. et al., 2004. Aberrant expansion of segmented filamentous bacteria in IgA-deficient gut. Proc. Natl. Acad. Sci. 101:1981), demonstrating one means by which the host immune system is influenced by the gut microbiota. Recent discoveries have also provided additional evidence that symbiotic bacteria influence the function of the specialized mucosal dendritic cells and IgA secretions, influencing the subsequent host intestinal immune response (Tezuka, H., et al., 2007 Regulation of IgA production by naturally occurring TNF/iNOS-producing dendritic cells. Nature 448:929). Previous evidence also suggests that it is the bacterial populations in the host GIT that direct luminal cell surface receptor glycosylation of intestinal epithelial cells, which also influence pathogenic adherence (Bry, L., et al., 1996. A model of host-microbial interactions in an open mammalian ecosystem. Science 273: 1380). Additionally, several other products of microbial fermentation have been shown to have effects including adenosine tri-phosphate (ATP) production (Atarashi, K. et al., 2008. ATP drives lamina propria T₈17 cell differentiation. Nature 455:808). Several other products of microbial fermentation as have also been shown to have immunomodulatory effects. Mice treated with antibiotics, followed by exposure to the parasite Encephalitizoan cuniculi and which were then treated with DNA isolated from normal gut bacteria, resulted in decreased parasite burden (Hall, J., et al., 2008. Commensal DNA limits regulatory T cell conversion and is a natural adjuvant of intestinal immune responses. 2008. Immunity. 29:637) These studies demonstrate that cellular constituents/inventive compositions alone may positively influence the host immune response, providing further evidence that cellular constituents may be beneficial to the host. More specifically, reconstitution of germ-free mice with bacterial populations that do not contain Bacteroidetes species fail to restore proper immune balance in the host (Ivonav, Il., et al., 2008. Specific microbiota direct differentiation of IL-17-producing T-helper cells in the mucosa of the small intestine. Cell Host Microbe 4:337), providing additional evidence that a species within the genus Bacteroides and/or cellular constituents/inventive compositions isolated from these bacteria could be utilized beneficially to support host health and modulate the inflammatory response and associated diseases.

Gastrointestinal microbiota play a key role in maintaining host and GI health as well as preventing disease. It appears that, in addition to bacterial attachment to host cell surface receptors, it is the molecular dialogue between the molecules produced by and/or constituents of the bacterial cells in conjunction with the host immune receptors that enable the microbiota to confer host resistance to disease. Thus, a composition consisting of one or more species from the genus Bacteroides, or a modified form thereof, a cellular constituent, or a derivative of a cellular constituent, including fragments therefrom, molecular complexes/networks therefrom, molecules therefrom, and/or synthetic or semi-synthetic analogs thereof, and/or mixtures of any of these, may be utilized to modulate any of the associated disease states, to the benefit of the host.

Accordingly, in various embodiments, the present invention is directed to cellular constituents, modified bacteria, compositions, and methods for modulating an inflammatory response and/or associated disease states. More specifically, in one embodiment, the invention is directed to a cellular constituent lysed from, produced by or isolated from a bacteria from the genus Bacteroides, or a derivative thereof, for example, a synthetically derived molecule that is based upon a molecule/molecular pattern from a species within the genus Bacteroides. In another embodiment, the invention is directed to a genetically modified form of bacteria from the genus Bacteroides. In another embodiment, the invention is directed to a composition including a cellular constituent from one or more bacteria from the genus Bacteroides, or a derivative thereof, or a genetically or chemically modified form of one or more bacteria from the genus Bacteroides.

Probiotic compositions comprising bacteria from the genus Bacteroides are described in U.S. patent application Ser. No. 12/255,152, filed Oct. 21, 2008, US 2009/0110664, which is incorporated herein by reference in its entirety.

Although the mechanisms have not been completely elucidated, evidence is available as to the co-relation between the microbiota and various disease states. Thus, the compositions according to the invention comprising one or more species from the genus Bacteroides, or a genetic or chemical modification thereof, or cell constituent thereof, or a derivative of such cell constituent, including molecular complexes/networks therefrom, molecules therefrom, and/or synthetic or semi-synthetic analogs thereof, including mixtures thereof, may be utilized to modulate inflammation, i.e., corporal or gastrointestinal inflammation in an individual, and, more specifically, to treat, delay the onset of, or reduce the symptoms of one or more inflammatory conditions/diseases, including corporal or gastrointestinal inflammation, and/or associated diseases such diabetes, Irritable Bowel Syndrome, Crohn's Disease, colitis, asthma, multiple sclerosis, cancer, including cancers such as colon, colorectal, prostate, bladder, lymphoma, hepatocellular carcinoma, peritoneal, lung, brain, sarcomas from bone, cartilege, muscle, fat or vascular tissues, bronchial, esophageal, thyroid, ovarian, breast, pancreatic, liver and gastric, rheumatoid arthritis, gingivitis, atopic diseases, including but not limited to hay fever, food allergies, eczema, rhinitis, dermatitis, conjunctivitis, atopic syndrome and keratosis pelaris, ocular inflammatory disease, strokes, hypertension, cardiovascular disease, depression, and atherosclerosis, and/or any of the associated disease states. Within the context of the present disclosure, delaying the onset of a disease or condition includes reducing a risk of developing a disease or condition. The methods comprise administering a composition according to the invention to an individual having or at risk of having such a disease.

Cellular constituents isolated or synthesized from the Bacteroides genus may be isolated and utilized to modulate the inflammatory response and thus decrease the effect or prevent the onset of the previously stated diseases and/or conditions. Cellular constituents and derivatives thereof include any molecule or molecules from a species of bacteria from the genus Bacteroides, symbiotic factors, cell wall constituents, molecules produced by the bacterial cells, cellular constituents/cell fragments therefrom, molecular complexes/networks therefrom, molecules therefrom, and/or synthetic or semi-synthetic analogs of these, including those prepared according to extreme biological synthetic techniques, and/or mixtures of any of these, which may be utilized to modulate inflammation, as described herein, and/or any of the associated disease states.

In one embodiment, the invention is directed to cellular components lysed from, produced by or isolated from, any species from the genus Bacteroides, or a derivative thereof. In another embodiment, the invention is directed to a genetically modified or extreme biological synthesized form of such bacteria or cellular component thereof.

Bacteria useful in the preparation of the disclosed cellular constituent preparation include, but are not limited to, any species in the Bacteroides genus such as Bacteroides thetaiotaomicron (ATTC29148), B. fragilis (NCTC9343), B. vulgatus (ATCC8482), B. distasonis (ATCC8503), B. ovatus, B. stercoris, B. merdae, B. uniformis, B. eggerithii, and B. caccae with B. fragilis as the type strain. In a specific embodiment, the bacteria is selected from the group consisting of Bacteroides thetaiotaomicron, B. fragilis, B. vulgatis, B. distasonis, B. ovatus, B. merdae, B. uniformis, B. eggerithii, and B. caccae.

In a specific embodiment, one or more cellular constituents according to the invention may be directed to appropriate intestinal epithelial cell surface receptors, decreasing the binding of pathogenic bacteria or pathogenic bacterial cellular constituents.

In another embodiment, the cellular constituent comprises a cell wall component, for example selected from the group consisting of lipopolysaccharides, proteins, carbohydrates, lipids, lipoproteins, glycoproteins, and combinations thereof. In another embodiment, the cellular material comprises DNA or RNA, for example 16S RNA, messenger RNA, ribosomal RNA, or the like.

In another embodiment, the cellular constituents comprise a molecule or molecules produced by a species within the genus Bacteroides.

In another embodiment, the cellular constituents are produced by de novo biological synthesis of any cellular constituents patterned after any bacterial species from the genus Bacteroides.

The cellular constituent composition may be provided as a single molecule or a combination of molecules, lysed from bacterial cells or synthetically derived from molecules obtained from a bacterial species from the genus Bacteroides or any combination thereof. Those skilled in the art will appreciate that the Bacteroides bacterial molecules may be lysed directly from the bacteria or synthetically manufactured based upon any molecular constituent of any Bacteroides species.

Examples of the cellular constituents, include, but are not limited to, cell fragments, molecular complexes or networks, cell wall constituents and/or unique products/molecules, by any species within the genus Bacteroides or any genetically modified species (including any de novo synthesis) which may include, but is not limited to, site mutations, insertion, deletion, or modification of genetic material from any source (viral, bacterial, human, etc.), synthetic or semi-synthetic analogs of any molecules and/or any products/molecules produced by any Bacteroides cells and/or genetically modified bacterial cells, as well as any synthetic or semi-synthetic analogs of any of these molecules from any species within this genus, as those skilled in the art will appreciate. Examples of processes by which such cellular constituents and modified bacteria may be obtained are provided. Additional processes will be evident to those of skill in the art in view of the present disclosure.

In one embodiment, the process for producing cellular constituents according to the invention begins with lysis of bacterial cells which results in disruption of the cell membrane and subsequent release of cellular contents (molecules, organelles, etc.). Methods of cellular lysis include, but are not limited to, mechanical (for example, blending), optical (for example, laser), chemical (for example, using surfactants such as sodium dodecyl sulfate), sonic (for example, sonication), electrical (for example, voltage), osmotic (for example, hypotonic solutions), or enzymatic (for example, lysozyme) processes. A common procedure comprises placing cells in to a Waring® blender with a suitable solution to mechanically disrupt the membrane. Alternatively, cells may be placed into a hypotonic solution which causes the membranes to burst. Cellular suspensions may also be forced through small spaces (liquid homogenization) resulting in disruption of the cell membranes. Once lysed, separation typically begins with gradient centrifugation procedures followed by separation techniques dependent upon the cellular component, followed by additional isolation and purification procedures. These procedures may include, but are not limited to, for example, extraction with gradient centrifugation utilizing various solutions, for example, phosphate buffer solutions, salt solutions or ammonium sulfate, and/or Soxhlet processes for separating proteins, ethanol for separating nucleic acids, and phenol for lipid soluble components. Additional procedures for further purification include, but are not limited to, dialysis and/or filtration/gel filtration and/or various forms of high performance/pressure liquid chromatography (HPLC) utilizing appropriate columns. Other methodologies may include, but are not limited to, various forms of electrophoresis such as sodium dodecyl sulfate polyacrylamide gel electrophoresis (SDS-PAGE), spectrophotometry, enzyme-linked immunosorbant assays (ELISA), fluorescence blots, and polymerase chain reaction for further separation, purification and identification/amplification, and/or for biological activity assays. Other methodologies which may also be employed include nucleic acid amplification by utilization of a cloning DNA vector and amplification (commonly referred to as recombinant DNA technology or genetic engineering). Those skilled in the art will appreciate that various techniques may be utilized for lysis and subsequent separation, identification and amplification and production of inventive compositions/cellular constituents for manufacture and biological assay purposes.

The cellular components, including their various conjugates, include but are not limited to, for example, proteins (endotoxins, transmembrane proteins, integral proteins and enzymes), glycoproteins, constituents of the periplasm, glycolipids, lipopolysaccharides (LPS), MAMPs/PAMPs, cell surface molecules (antigens, adhesions, etc.), cytoplasmic molecules or products, lipoproteins, porins, peptidoglycans, carbohydrates, peptides, lipid A, O polysaccharides, phospholipids, lipids, or genetic components such as DNA, RNA and nucleic acids. In one specific embodiment, the cellular constituent comprises LPS (lipopolysaccharides), DNA/RNA/nucleic acids, O polysaccharides, lipid A, endotoxins, and/or MAMPs. In another embodiment, the cellular constituent comprises O polysaccharide and/or lipid A.

In addition, cellular constituents include but are not limited to unique molecules produced and secreted by any bacteria from the genus Bacteroides such as proteins, carbohydrates, lipids, and combinations or derivatives thereof, plasmids, nucleic acids, antibiotics and bacteriocins, through any system, including but not limited to ABC-transporters (ATP-binding cassette transporters including but not limited to Types I-VI), metabolic products, and release of outer membrane molecules which contain but are not limited to, for example, periplasmic or cytoplasmic materials.

The cellular constituents include but are not limited to synthetic or semi- synthetic analogs of any of the cellular constituents described previously, including but not limited to the pharmacophore (frequently utilized to refer to the active site of a compound which is the molecular structure which interacts with the receptor, producing the desired result) or the auxophore (molecular components which are not part of the active site but which result in modulation of biological activity if modified).

Derivatives of the described cellular constituents are also encompassed by the present invention. These derivatives may comprise modifications including, but not limited to, addition, removal or alteration of atoms within a molecule and/or addition, removal or alteration of one or more molecules within a molecular network/complex or addition or excision of atoms/molecules or groups of molecules. For example, the addition of an ethyl group or a hydroxyl, substitution of a hydroxyl group with an amine, modifying functional groups, for example by substitution of a thiol with a methyl group, substitution for example of an oxygen atom with sulfur, or any molecular substitution or alteration of a stereogenic center to form a new stereoisomer, alteration of backbone configuration to form a new isomer, or any other alteration where a specific structural or chemical change results in a modulation of activity or potency, are included. Additions to the cellular constituent structure include, for example lengthening of a saturated carbon chain from one to five atoms (methyl to pentyl) or longer, or addition of a methylamino group, chain branching, ring modification or maneuvering of the position of a group, for example amino or sulfonyl groups from ortho to para, which may result in improved biological activity/host response. Synthetic analogs include homologation of the molecular structure, for example any group of molecules that differ by one constant unit, for example CH₂₋ and transformation of the backbone or substituent groups from linear to cyclic or vice versa (for example modifications of ringed amino acids or ringed structure of nucleic acids). Synthesis or derivatives of cellular constituents include modifying groups with isosteric groups to form bioisosteres (a chemical functional group replaced by another chemical group resulting in similar bioactivity) which have chemical or physical similarities as well as similar biological activity. For example, this includes, but is not limited to, molecules with similar numbers of valence electrons or those which do not have the same number of atoms but have similar peripheral layers of electrons. These include but are not limited to univalent atoms such as chlorine, fluorine or the hydroxyl group, bivalent atoms such as oxygen and selenium, and ring equivalents such as benzene or thiophene. Nonclassical bioisosteres which do not have similar numbers of atoms or valence electrons but do have similar biological activity include, but are not limited to, modifications to the carbonyl group or carboxyl group or heterocyclic aromatic groups such as oxazoles, thiophenes, imidazoles, etc. Those skilled in the art will appreciate that this small list is only an illustration of several of the various specific embodiments encompassed within the present invention.

Quantities of appropriate Bacteroides bacteria may be generated using a fermentation process. For example, a sterile, anaerobic fermentor may be charged with media, such as glucose, polysaccharides, oligosaccharides, mono- and disaccharides, yeast extract, protein/nitrogen sources, macronutrients and trace nutrients (vitamins and minerals), and cultures of the desired Bacteroides bacteria may be added to the media. During fermentation, concentration (colony forming units per gram), purity, safety and lack of contaminants may be monitored to ensure a quality end result. After fermentation, the Bacteroides bacteria cells may be separated from the media using various well known techniques, such as filtering, centrifuging and the like and the cellular constituents lysed and/or separated from other cellular constituents. The separated cellular constituents may be dried by, for example, lyophilization, spray drying, heat drying or combinations thereof, with protective solutions/media added as needed.

In another embodiment, the cellular constituents are produced by de novo biological synthesis of any cellular constituents patterned after any bacterial species from the genus Bacteroides.

A genetically modified bacterium from the genus Bacteroides suitable for use in the present invention consists of any genetic change including but not limited to a specific change in a gene (site-directed mutagenesis), genetic modification by insertion or deletion of a particular gene (utilizing restriction enzymes) and/or a plasmid (for example R factor plasmids) or virus (for example shuttle viruses), addition of any genetic material from any source (viral, animal, plant, yeast, etc.), and covalent modification of nucleotides/genes/genomes which result in a change within the cells themselves or molecules/products of the bacterial cells.

The present invention also relates to compositions containing the disclosed cellular constituents, or derivative thereof, Bacteroides bacteria, or genetically modified form thereof, such compositions referred to herein as inventive compositions, and to methods employing such compositions as described herein.

The compositions of cellular constituent or derivative thereof as described, bacteria or genetically modified form thereof may begin with an appropriate medium to which an appropriate protectant may be added for molecular protection. Examples of appropriate protectants include, but are not limited to, distilled water, polyethylene glycol, sucrose, trehalose, skim milk, xylose, hemicellulose, pectin, amylose, amylopectin, xylan, arabinogalactan, starch (e.g., potato starch or rice starch) and polyvinylpyrrolidone.

In another embodiment, the disclosed cellular constituent composition may include a quantity of the bacterial cellular constituents and, optionally, one or more physiologically acceptable carriers. In a specific embodiment, the carrier is a pharmaceutically acceptable carrier and the composition is adapted for administration to a human or other animal. The carrier may be provided to facilitate delivery to a subject animal in need thereof. As used herein, the term “carrier” is intended to broadly refer to any substance (e.g., a tableting agent or a liquid) or article (e.g., a capsule shell or a polymer matrix) that facilitates administration of the Bacteroides compositions by providing a medium for their conveyance to the consuming animal. Those skilled in the art will appreciate that the carrier should not significantly inhibit the intended cellular constituent value to the subject. As set forth in further detail below, administration may be by any desired route, including oral, injection, inhalation, topical, or other known administration route.

The inventive compositions comprising Bacteroides bacteria and/or the Bacteroides bacterial cellular constituents may be prepared in various forms for administration, such as capsules, suppositories, tablets, food/drink, inhalant, sublingual fluid, lotion, eye drops or ear drops and the like. In another aspect, the inventive compositions may be provided as a semi-solid or cake or in powdered form. In one embodiment, optionally, the inventive compositions may include various pharmaceutically acceptable excipients, such as microcrystalline cellulose, mannitol, glucose, defatted milk powder, polyvinylpyrrolidone, starch or combinations thereof, and/or any of the excipients mentioned herein.

The present disclosure provides a cellular constituent composition from any appropriate species of bacteria from the genus Bacteroides, as well as a system and method for using the disclosed cellular constituent(s) composition to treat, delay the onset of, including to reduce the risk of developing, and/or reduce the symptoms of a disease or condition of one or more gastrointestinal or systemic inflammatory conditions or one or more inflammatory conditions/diseases, including corporal or gastrointestinal inflammation, and/or associated diseases such diabetes, Irritable Bowel Syndrome, Crohn's Disease, colitis, asthma, multiple sclerosis, cancer, including cancers such as colon, colorectal, prostate, bladder, lymphoma, hepatocellular carcinoma, peritoneal, lung, brain, sarcomas from bone, cartilage, muscle, fat or vascular tissues, bronchial, esophageal, thyroid, ovarian, breast, pancreatic, liver and gastric, rheumatoid arthritis, gingivitis, atopic diseases, including but not limited to hay fever, food allergies, eczema, rhinitis, dermatitis, conjunctivitis, atopic syndrome and keratosis pelaris, ocular inflammatory disease, strokes, hypertension, cardiovascular disease, depression, atherosclerosis, or rheumatoid arthritis, and/or any of the associated disease states, in animals, such as humans, horses, rats, mice, ruminants, primates, monkeys, hamsters, rabbits, dogs, cats and various avian and fish species. The disclosed cellular compositions as described herein, the “inventive compositions”, may be delivered to the host to decrease, delay or reduce the symptoms of gastrointestinal or systemic inflammation of the previously mentioned conditions. In a specific embodiment, the methods are practiced in humans.

In one embodiment, the cellular constituent and or the inventive composition is provided in lyophilized form in accordance with conventional techniques. An example of an appropriate lyophilization process may begin with a media carrying appropriate carriers including, but not limited to, one or more protectants, buffers, stabilizers, and, more specifically, on or more of distilled water, polyethylene glycol, sucrose, trehalose, skim milk, xylose, hemicellulose, pectin, amylose, amylopectin, xylan, arabinogalactan, starch (e.g., potato starch or rice starch), polyvinylpyrrolidone, iron oxide, polydextrose, polyvinyl acetate phthalate, propylene glycol, shellac wax, sodium alginate, sodium bicarbonate, triethyl citrate, lactose, mannitol, sorbitan, sodium phosphates, sorbitol, dimethicone, sodium lauryl sulfate, croscarmellose sodium, lecithin, and xantham gum.

In one embodiment, the inventive compositions may be provided in a sustained-release (SR), extended release (ER, XR, or XL), time-release controlled-release (CR) or continuous release (CR or Contin) form, for example, in a tablet, soft gel, suppository or capsule form, in order to release the molecules over an extended period of time. These constituents may be embedded in a matrix of insoluble substances and/or conventional additives, which include, but are not limited to, acrylics, chitin, polymers, a soluble fiber that swells to form a gel or matrix, an insoluble fiber, microcrystalline cellulose, propyl gallate, coloring agents and/or hypromellose. In a specific embodiment, the sustained-release, extended release, time-release controlled-release or continuous release form is for oral administration

In a specific embodiment, the cellular constituent(s) or bacteria are delivered in a timed release, extended release or sustained release form. Examples of appropriate formulation components include, but are not limited to, one or more of hyprocellulose, microcrystalline cellulose, magnesium stearate, milk proteins, titanium dioxide, sodium citrate, propyl gallate, riboflavin, inulin, iron oxide, silical, silicon dioxide, magnesium silicate, maltodextrin, chlorophyll, potato starch, calcium phosphate, sodium starch glycolate, tumeric, carbonate, carnuba wax, triacetin, polysorbate 80, methylacrylic acid copolymer, chitin, acrylics, prop-2-enoyl, acrylyl, acryl, povidone, and stearic acid.

In one aspect, the disclosed cellular constituent composition/inventive compositions may be prepared as a capsule/soft gel. The capsule (i.e., the carrier) may be a hollow, generally cylindrical capsule formed from various substances, such as gelatin, cellulose, carbohydrate, hypromellose or the like. The capsule may receive the Bacteroides bacteria or cellular constituents/inventive composition therein. Optionally, and in addition to the appropriate Bacteroides bacteria or cellular constituents/inventive composition, the capsule may include but is not limited to coloring, flavoring, rice or other starch, glycerin, and/or titanium dioxide.

In a second aspect, the inventive compositions may be prepared as a suppository. The suppository may include but is not limited to the appropriate Bacteroides bacteria or cellular constituent and one or more carriers, such as polyethylene glycol, acacia, acetylated monoglycerides, carnuba wax, cellulose acetate phthalate, corn starch, dibutyl phthalate, docusate sodium, gelatin, glycerin, iron oxides, kaolin, lactose, magnesium stearate, methyl paraben, pharmaceutical glaze, povidone, propyl paraben, sodium benzoate, sorbitan monoleate, sucrose talc, titanium dioxide, white wax and coloring agents.

In a third aspect, the inventive compositions may be prepared as a tablet. The tablet may include the appropriate Bacteroides bacteria or cellular constituent/inventive composition and one or more tableting agents (i.e., carriers), such as dibasic calcium phosphate, stearic acid, croscarmellose, silica, cellulose and cellulose coating. The tablets may be formed using a direct compression process, though those skilled in the art will appreciate that various techniques may be used to form the tablets.

In a fourth aspect, the disclosed inventive compositions may be formed as food or drink or, alternatively, as an additive to food or drink, wherein an appropriate quantity of Bacteroides bacteria or cellular constituent(s) is/are added to the food or drink to render the food or drink the carrier. In a specific embodiment, the inventive compositions are an additive to chewing gum, lozenges, hard or soft candy, or the like.

In a fifth aspect, the inventive compositions may be provided in a sublingual fluid which may contain but is not limited to one or more components selected from water, sorbitol, glycerin, citric acid, potassium sorbate and flavoring.

In a sixth aspect, the inventive compositions may be provided in a mouth wash which may include but is not limited one or more components selected from to water, ethanol, sorbitol, poloxamer 407, benzoic acid, flavoring, sodium saccharin, sodium citrate, citric acid, and food safe dyes.

In a seventh aspect, the inventive compositions may be provided in a pressurized meter-dosed inhaler. Such an inhaler may include a pressurized carrier, for example, which may include but is not limited to 1,1,1,2-tetraflouroethane (HFA-134A), etc.

In an eighth aspect, the inventive compositions may be provided in an eye drop solution which may include but is not limited to one or more components selected from benzylkonium chloride, disodium edetate, potassium chloride, water, sodium bicarbonate, sodium citrate, sodium chloride, sodium phosphate (mono- and dibasic), polyvinyl alcohol, povidine, nonanoyl EDTA, polyquaternium-1, and myristamidoproyl dimethylamine.

In a ninth aspect, the inventive compositions may be provided in ear drops which may include but is not limited to one or more components selected from benzylkonium chloride, glycerin and water.

In a tenth aspect, the inventive compositions may be provided in a lotion which may include but is not limited to one or more components selected from water, glycerin, petrolatum, cetearyl alcohol, dimethicone, fragrance, ceteareth-20, sodium hydroxide, methylparaben, propylene glycol, diazolinodyl urea, disodium EDTA, propylparaben, distearyldimonium chloride, glyceryl laurate, potassium hydroxide, behentrimonium methosulfate, cocamiopropyl PG-dimonium chloride phosphate, octyldodecanol, and PEG-100 stearate.

The concentration of the Bacteroides bacteria or cellular constituents in the inventive compositions may vary depending upon the desired result, the type and form of bacteria or cellular constituent used, the form and the intended method of administration, among other things. For example, an inventive composition may be prepared having a concentration of bacteria or cellular constituents in the preparation of no less than about 1 mg to about 1 g by weight, or 1-30X HPUS (Homeopathic Pharmacopia of the US) based upon the total weight of the preparation. In one embodiment, the compositions may be administered one, two, three, or more times daily. In another embodiment, the compositions are administered every 4-6 hours. In yet another embodiment, the compositions are administered one, two, three or more times weekly.

Specific examples of suitable compositions contemplated for use in the present invention are provided below.

### Example 1

This example shows the preparation of cellular constituents for used in therapeutic compositions.

Bacterial cell cultures are grown in large vats under tightly controlled conditions. A cellular constituent, for example a protein, is obtained from the bacterial cells themselves by cellular lysis, extraction and purification, or, alternatively, from bacterial cell secretions obtained by stimulation of the bacteria to produce the protein, for example, by varying conditions such as pH, temperature, oxygen, nutrient or other variable. Sterile glass cultures/tubes are then inoculated with the medium containing the lysed cells and/or protein, and the suspension media, including, but not limited, for example, up to 10% skim milk, with or without 5% sodium gluconate. The material is then, for example, subjected to centrifugation and washing with the appropriate sterile medium (for example, suspension media or buffer solution). Conventional additives for freeze drying, including protectants, stabilizers, buffers, and the like may be added. Fluids are typically removed prior to freeze drying (lyophilization), which may be conducted, for example, at temperatures of from about −20° C. to approximately −200° C., more specifically, in a range of −50° C. to −80° C., or lower, typically under a vacuum and for several hours. Once drying is complete, inert or inactive ingredients, etc., are added, including, but not limited to rice powder, magnesium stearate, dicalcium phosphate, cellulose, stearic acid, calcium carbonate and/or silicon dioxide, to provide a dry powder formulation.

### Example 2

This example shows a suitable capsule product.

Using a lyophilization process, a quantity of cellular constituents from B. thetaiotaomicron cells is prepared in powdered form (“Active Ingredient 1”) using a procedure as described in Example 1.

TABLE 1

| No. | Ingredient | mg/Capsule |
|---|---|---|
| 1 | Active Ingredient 1 | 200 |
| 2 | Lactose USP | 180 |
| 3 | Corn Starch, Food Grade | 60 |
| 4 | Magnesium Stearate NF | 10 |

Components 1-4 from Table 1 are mixed in a suitable mixer for 10 minutes. After mixing, 450 milligrams of the mixture is charged into a two-piece gelatin or hypromellose capsule and the capsule is sealed.

### Example 3

This example shows a suitable tablet product.

Using a lyophilization process as described in Example 1, a quantity of cellular constituents from B. uniformis cells is prepared in powdered form (“Active Ingredient 2”).

TABLE 2

| No. | Ingredient | mg/Tablet |
|---|---|---|
| 1 | Active Ingredient 2 | 65 |
| 2 | Microcrystalline Cellulose | 135 |
| 3 | Glucose | 250 |

Components 1-3 from Table 2 are mixed in a suitable mixer for 10 minutes. The mixture is then compressed into 450 milligram tablets using a tableting press.

### Example 4

This example shows a suitable suppository product.

Using a lyophilization process as described in Example 1, a quantity of cellular constituents from B. vulgatus cells is prepared in powdered form (“Active Ingredient 3”)

TABLE 3

| No. | Ingredient | g/dosage |
|---|---|---|
| 1 | Active Ingredient 3 | 15 |
| 2 | Cacao Butter | 30 |
| 3 | Yellow Wax | 5 |
| 4 | Petroleum Jelly | 5 |
| 5 | Sodium Stearate | 3 |

Components 2-4 from Table 3 are charged into a suitable mixer heated to a temperature of 60° C. while constantly stirring to form a first mixture. Separately, Components 1 and 5 from Table 3 are charged into a mixer and mixed for 10 minutes to form a second mixture. Slowly, and while stifling, the second mixture is added to the first mixture and the resulting mixture is continuously stirred for 10 minutes and then poured into pre-formed suppository shells. The filled suppository shells are allowed to cool until the suppositories set.

The disclosed inventive compositions may be administered to a subject to treat, delay the onset of, and/or or reduce the symptoms of inflammation and associated diseases. Furthermore, the disclosed cellular constituent compositions may be used to sustain beneficial effects pursuant to an appropriate maintenance protocol.

Furthermore, in an option embodiment, the present methods may employ a cleansing step prior to administration of the inventive composition. Alternatively, cleansing of the gut may not be utilized prior to administration. Those skilled in the art will appreciate that any medically approved chemical/solution that induces diarrhea may be used as a cleansing chemical/solution for such a step. Examples of appropriate cleansing chemicals/solutions include, without limitation, magnesium citrate, sodium phosphate, dibasic (any form), sodium phosphate, monobasic, any form, potassium phosphate, monobasic, any form, and potassium phosphate, dibasic, any form. After the gastrointestinal tract has been cleansed, the disclosed inventive composition may be administered. An appropriate inventive composition administration schedule may include, for example, administration of a certain number of cellular constituent compositions (e.g., 3 capsules) with each meal for a certain number of days (e.g., for three days). However, those skilled in the art will appreciate that the quantity and frequency of administration of the disclosed inventive compositions may depend upon the type of bacteria or bacterial cellular constituents being administered, the concentration of bacteria or cellular constituents in the composition, and the weight, height and/or age of the subject, among other things.

Beneficial effects may be sustained by continued administration of the disclosed inventive compositions (e.g., one capsule per day or one capsule with each meal) together with a proper maintenance program. For example, a subject may be advised to avoid foods that are high in fat and sugar and focus on consuming a certain quantity of fruits and vegetables (e.g., two fresh fruits and two vegetables every day). Furthermore, a subject may be advised to undergo a minimum three sessions of 30 minutes of moderate exercise, such as brisk walking, each week. More fresh fruits and vegetables and more exercise should be encouraged.

To encourage proper use of the disclosed inventive compositions, the inventive compositions may be provided together with instructions for use, and/or suggested cleansing/inoculation and inoculation/maintenance protocols, and/or a covenant that a user may customize and use to track progress. The instructions and/or covenant may be provided together with the inventive composition compositions, compositions in a kit or bundle.

Accordingly, those skilled in the art will appreciate that the disclosed cellular constituents, inventive compositions, and associated methods may be used to aid the anti- inflammatory system without the need for invasive surgeries or other drastic techniques by increasing the populations of beneficial bacterial species or cellular constituents in the gastrointestinal tract and other systems exposed to the external environment. The beneficial bacterial species or cellular constituents may be sustained with continued administration of the inventive composition and, optionally, an appropriate maintenance regimen, including proper diet and exercise.

The specific embodiments and examples set forth in the present specification are illustrative in nature and are not limiting of the scope of the invention defined by the present claims. Although various aspects of the disclosed cellular constituents, inventive compositions, and methods may occur to those skilled in the art upon reading the specification, the present invention includes such modifications and is limited only by the scope of the claims.

The following references are cited herein and/or may be relevant to one or more aspects discussed in the specification:

Alberti, K. G. and P .Z. Zimmei. 1998. Definition, diagnosis and classification of diabetes mellitus and its complications, part 1: diagnosis and classification of diabetes mellitus, provisional report of a WHO consultation. Diab. Med. 15:539-553.
Atarashi, K., J Nishimura, T. Shima, Y Umesaki, M. Yamamto, M. Onoue, H. Yagita, N, Ishii, R. Evens, K. Honda and K. Takeda. 2008. ATP drives lamina propria T₈17 cell differentiation. Nature 455:808-814.
Bals, R., M. J. Goldman and J. M. Wilson. 1998. Mouse beta-defensin 1 is a salt-sensitive antimicrobial peptide present in epithelia of the lung and urogenital tract. Infect. and Immun.66 (3):1225-1232.
Bjorksten, B. 1999. The environmental influence on childhood asthma. Allergy. 54:17-23.
Brasier, A. R. 2006. The NF-kB regulatory network. Cardiovasc. Toxicol. 6(2): 111-130.
Bry, L., P. G. Falk, T. Midvelt and J. L. Gordon. 1996. A model of host-microbial interactions in an open mammalian ecosystem. Science 273: 1380-1383.
Cerutti, A. 2008. The regulation of IgA class switching. Nature Rev. Immunol. 8:421-434.
Corti, R., R. Hutter, J. J. Badimon, J. J. Badimon and V. Fuster. 2004. Evolving concepts in the triad of atherosclerosis, inflammation and thrombosis. J. Thromb. Thromolysis. 17:35-44.
De Winter, H., H. Cheroutre and M. Kronenberg. 1999. Mucosal immunity and inflammation. II. The yin and yang of T cells in intestinal inflammation: pathogenic and protective roles in a mouse colitis model. Am. J. Physiol. 276:G1317-G1321.
Doyle, S. L. and L. A. O'Neill. 2006. Toll-like rececptors; from the discovery of NF-kB to new insights into transcriptional regulations in innate immunity. Biochem. Pharmacol. 72(9):1102-1113.
Elson, C. O. Y. Cong, C. T. Weaver, T. R. Schoeb, T. K. McClanahan, R. B. Fick and R. A. Kastelein. 2007. Monoclonal anti-interleukin 23 reverses active colitis in a T cell-mediated model in mice. Gastroenterology 132:2359-2370.
Frank, D., N. A. L. Amand, R. A. Feldman, E. C. Boedeker and N. R. Pace. 2007. Molecular-phylogenetic characterization of microbial community imbalances in human inflammatory bowel diseases. Proc. Natl. Acad. Sci. USA. 104(34):13780-13785.
Garrett, W. S., G. M. Lord, S. Punit, G. Lugo-Villarino, S. K. Mazmanian, I Susumu, J. N. Glickman and L. H. Gilmscher. 2007. Communicable ulcerative colitis induced by T-bet deficiency in the innate immune system. Cell. 131:33-45.
Gilmore, T. D. 1999. The Rel/NF-kB signal transduction pathway: introduction. Oncogene: 18(49):6842-6844.
Gilmore, T. D. 2006. Introduction to NF-kB: players, pathways, perspectives. Oncogene: 25(51):6680-6684.
Hall, J., N. Bouladoux, C. M. Sun, W. A. Wohlfert, R. B. Blank, Q. Zhu, M. E. Grigg, J.A. Berofsky and Y. Belkaid. 2008. Commensal DNA limits regulatory T cell conversion and is a natural adjuvant of intestinal immune responses. 2008. Immunity. 29:637-649.
Halpert, I., U. I. Sirest, J. D. Roby, U. I. Sires, S. Potter-Perigo, T. N. Wight, S .D. Shapiro, H. G. Welgus, S. A. Wickliff and W. C. Parks. 1996. Matrilysin is expressed ny lipid-laden macrpophages at sites of potential rupture in atherosclerotic lesions and localizes to areas of versican deposition, a proteolytic substrate for the enzyme. Proc. Natl. Acad. Sci. USA. 93:9748-9753.
He, B., W. Xu, P. A. Santini, A. D. Polydorides, A. Chiu, J. Estrella, M. Shan, A. Chadburn, M. Shan, A. Chadburn, V. Villanacci, A. Plebani, D. M. Knowles, M. Rescigo and A. Cerutti. 2007. Intestinal bacteria trigger T cell independent immunoglobulin A₂ class switching by inducing epithelial-cell secretion of the cytokine APRIL. Immunity 26:812-826.
Henderson, B., S. Poole and M. Wilson. 1996. Bacterial modulins: a novel class of virulence factors which cause host tissue pathology by inducing cytokine synthesis. Microbiol. Rev. 60(2):316-341.
Hooper, L. V., M. H. Wong, A. Thelin, L. Hansson, P. G. Falk and J .I. Gordon. 2001. Molecular analysis of commensal host-microbial relationships in the intestine. Science 291:881-884.
Hotamisligil, G. S. 2006. Inflammation and metabolic disorders. Nature. 444:860-867.
Hu, F. B., J. E. Manson, M. J. Stamper, G. Colditz, S. Lui, C. G. Soloman and W. C. Willet. 2001. Diet, Lifestyle and the risk of type 2 diabetes mellitus in women. New Engl. J. Med. 345(11):790-797.
Hultgren, S. J. S. Abraham, M. Caparon, P. Faulk, J. W. St. Geme III and S. Numark. 1993. Pilus and nonpilus bacterial adhesins:assembly and function in cell adhesion. Cell. 73:887-901.
Ivonav, I. I., R. deLlanos Frutos, N. Manel, K. Yoshinaga, D. B. Rifkin, B. Brett Finlay and D. R. Littman. 2008. Specific microbiota direct differentiation of IL-17-producing T-helper cells in the mucosa of the small intestine. Cell Host Microbe 4:337-349.
Kalliomaki, M. and E. Isolauri. 2002. Pandemic of atopic diseases- a lack of microbial exposure in early infancy? Curr. Drug Targets Infect. Disord. 2:193-199.
Kalliomaki, M. and E. Isolauri. 2003. Role of the intestinal flora in the development of allergy. Curr. Opin. Allergy Clin. Immunol. 3:15-20.
Kojima, S., T. Funshashi, H. Maryoshi, O. Honda, S. Sugiyama, H. Kawano, H. Soejima, S. Miyamoto, J. Hokamaki, T. Sakamoto, M. Yoshimura, A. Kitagawa, Y Matsuzawa and H. Ogawa. 2005. Levels of the adipocyte-derived plasma protein, adiponectin, have a close relationship with atheroma. Thromb. Res. 115:483-490.
Lepage, P., P. Seksik, M. Sutren, M.-F. de la Cochetiere, R. Jian, P. Mateua and J. Dore. 2005. Biodiversity of the mucosa-associated microbiota is stable along the distal digestive tract in healthy individuals and patients with IBD. Inflamm. Bowel Dis. 11:473-480.
Lin, W- W. and M. Karin. 2007. A cytokine-mediated link between innate immunity, inflammation and cancer. J. Clin. Invest. 117(5):1175-1183.
Lopez-Baodo, Y. S., C. L. Wilson, L. V. Hooper, J. I. Gordon, S. J. Hultgren and W. C. Parks. 2000. Bacterial Exposure induceds and activates matrilysin in mucosal epithelial cells. J. Cell Biol. 148(6):1305-1315.
Lupton, J. R. 2004. Microbial degradation products influence colon cancer risk: the butyrate controversy. J. Nutr. 134:479-482.
Macpherson, A. J. and T. Urh. 2004. Induction of protective IgA by intestinal dendritic cells carrying commensal bacteria. Science: 303:1662-1665.
Mazmanian, S. K., C. H. Lui, A. O. Tzianabos and D. Kasper. 2005. An immunomodulatory molecule of symbiotic bacteria directs maturation of the host immune system. Cell. 122:107-118.
Mazmanian, S. K., J. L. Round and D. Kasper. 2008. A microbial symbiosis factor prevents intestinal inflammatory disease. Nature. 453:620-625.
Murphy, G., M. I. Cockett, R. V. Ward and A. J. P. Docherty. 1991. Matrix mettaloproteinase degradation of elastin, type IV collagan and proteoglycan. A quantitative comparison of the activities of 95 kDa and 78 kDa gelatinases., stromylesins-1 and -2 and punctuated metalloproteinases (PUMP). Biochem. J. 277:277-279.
Muta, T and K. Takeshige. 2001. Essential roles of CD14 and lipopolysaccharide-binding protein for activation of toll-like receptor (TLR)2 as well as TLR4: Reconstitution of TLR2- and TLR4-activation by distinguished ligands in LPS preparations. Eur. J. Biochem. 268(16):4580-4589.
Nelson, D. E, A. E. Ihekwaba, E. M. Johnson, C. A. Gibney, B. E. Foreman, G. Nelson, S Vee, C. A. Horton, D. G. Spiller, S. W. Edwards, H. P. McDowell, J. F., J. F. Unitt, S. E. Sullivan, R. Grimley, N. Benson, D. Broomhead, D. B. Kell and M. R. White. 2004. Oscillations in NF-kB signaling control the dynamics of gene expression. Science. 306(5696):704-708.

Noverr, M. C. and G. B. Huffnagie. 2004. Does the microbiota regulate immune responses outside the gut? Trends Microbial. 12:562-568.
Ouettlette, A. J., M. M. Hsieh, M. T. Nosek, D. F. Cano-Gauci, K. M. Huttner, R. N. Buick, and S. E. Selsted. 1994. Mouse Paneth cell defensins: primary structures and antibacterial activities of numerous cryptdin isoforms. Infect. Immun. 62:5040-5047.
Ouellette, A. J. and S. E. Selsted. 1996. Paneth cell defensins: endogenous peptide components of intestinal cell defense. FASEB J. (Fed. Am. Soc. Exp. Biol. J.). 10:1280-1289.
Penders, J., C. Thijs, P. A. van den Brandt, I. Kummeling, B. Snijders, F. Stelma, H. Adams, R. von Ree and E. E. Stobberingh. 2007. Gut microbiota composition and development of atopic manifestations in infancy: the KOALA Birth Cohort Study. Gut. 56:661-667.
Peterson, D. A., N. P. McNutty, J. L. Guruge and J. I. Gordon. 2007. IgA response to symbiotic bacteria as a mediator of gut homeostasis. Cell Host Microbe 2:328-339.
Podolsky, D. K. 2002. The current future understanding of inflammatory bowel disease. Best Pract. Res. Clin. Gastroenterol. 16:933-943.
Rakoff-Nahoum, S., J. Paglino, F. Eslami-Varzaneh, S. Edberg and R. Medzhitov. 2004. Recognition of commensal bacteria by Toll-like receptors is required for intestinal homeoastasis. Cell 118:229-241.
Ryo, M., T. Nakamura, S. Kihara, M. Kumada, S. Shibazaki, M. Takhashi, M. Nagai, Y. Matzuzawa and T. Funahashi. 2004. Adiponectin as a biomarker of the metabolic syndrome. Circ. J.68:975-981.
Saarialho-Kere, U. K., H. G. Welgus and W. C. Parks. 1993. Divergent mechanisms regulate interstitial collagenase and 92 kDa gelatinase expression in human monocyte-like cells exposed to bacterial endotoxin. J. Biol. Chem.(268):17354-17361.
Scanlan, P. D., F. Shanahan, C. O. Mahony and J. R. Marchesi. 2006. Culture-independent analyses of temporal variation of the dominant fecal microbiota and targeted bacterial subgroups in Crohn's disease. J. Clin. Microbiol. 44:3980-3088.
Shanahan, F. 2002. Crohn's Disease. Lancet. 359:62-69.
Shoelson, S. E., J. Lee and A. B. Goldfine. 2006. Inflammation and insulin resistance. J. Clin. Invest. 116:1793-1801.
Simpson, S. J., Y. P. de Jong, M. Comiskey, C. Terhorst. 2000. Pathways of T cell pathology in models of chronic intestinal inflammation. Int. Rev. Immunol. 19:1-37.
Sires, U. I., G. L. Griffin, T. Broekelman, R. P. Mecham, G. Murphy, A. E. Chung, H. G. Welgus and R. M. Senior. 1993. Degradation of entactin by matrix metaloproteinases. Susceptibility to matrylisin and identification of cleavage sites. J. Biol. Chem.268:2069-2074.
Skurk, T. and H. Hauner. 2004. Obesity and impaired fibrinolysis: role of adipose production of plasminogen activator-1. Int. J. Obes. 28:1357-1364.
Solinas, G, C. Vilcu, J. G. Neels, G. K. Banyopadhiyay, J- L Luo, W. Naugler, S. Grivnenikov, A. Wynshaw-boris, M. Scadeng, J. M. Olefsky and M. Karin. 2007. JNK1 in hematopoetically derived cells contributes to diet-induced inflammation and insulin resistance without affecting obesity. Cell Metabol. 6:386-397.
Suzuki, K., B. Meek, Y. Doi, M. Muramatsu, T. Chiba, T. Honjo and S. Fagarasan. 2004. Aberrant expansion of segmented filamentous bacteria in IgA-deficient gut. Proc. Natl. Acad. Sci. 101:1981-1986.
Targan, S. R. and L. C. Karp. 2005. Defects in mucosal immunity leading to ulcerative colitis. Immunol. Rev. 206:296-305.
Tezuka, H., Y. Abe, M. Iwata, H. Takeuchi, H. Ishikawa, M. Matsushita, T. Shiohara, S. Akira and T. Ohteki. 2007 Regulation of IgA production by naturally occurring TNF/iNOS-producing dendritic cells. Nature 448:929-933.
White, C. R. 2003. Insulin signaling in health and disease. Science.302:1710-1711.
Wilson, C. L., A. J. Ouellette, D. P. Satchell, T. Ayube, Y. S. Lopez-Boada, J. L, Stratman, S. J. Hultgren, L. M. Matrisian and W. C. Parks. 1999. Regulation of intestinal alpha-defensin activation by the metalloproteinase matrilysin in innate host defense. Science. 286:(113-123).
Wilson, M. R. Seymour and B. Henderson. 1998. Bacterial perturbation of cytokine networks. Infect. Immun. 66:2401-2409.
Zaneveld, J., P. J. Turnbaugh, C. Laozupone, R. E. Ley, M. Hamady, J. I. Gordon and R. Knight. 2008. Host-bacterial coevolution and the search for new drug targets. Curr. Opin. Chem. Biol. 12:109-114.

1. A cellular constituent lysed from, produced by, or isolated from a bacteria from the genus Bacteroides, or a derivative of said cellular constituent.

2. A cellular constituent or derivative thereof according to claim 1, wherein the cellular constituent is from a bacteria selected from the group consisting of Bacteroides thetaiotaomicron, B. fragilis, B. vulgatis, B. distasonis, B. ovatus, B. merdae, B. uniformis, B. eggerithii, and B. caccae.

3. A cellular constituent or derivative thereof according to claim 1, comprising DNA or RNA.

4. A cellular constituent or derivative thereof according to claim 1, comprising a cell wall component selected from the group consisting of lipopolysaccharides, lipids, carbohydrates, proteins, lipoproteins, glycoproteins, and combinations thereof.

5. A cellular constituent or derivative thereof according to claim 1, comprising a product of the bacteria and selected from the group consisting of lipids, carbohydrates, proteins and genetic material.

6. A food or drink supplemented with a cellular constituent or derivative thereof according to claim 1.

7. A composition for oral administration, comprising a cellular constituent or derivative thereof according to claim 1, and a physiologically acceptable carrier.

8. A composition according to claim 7, wherein the carrier is selected from the group consisting of a capsule shell, a tableting agent and a polymer matrix.

9. A composition according to claim 7, wherein the carrier is selected from the group consisting of a capsule shell, a tableting agent, a polymer matrix, and a component providing extended release, delayed release or sustained release of the cellular constituent or derivative thereof.

10. A method of treating, delaying the onset of, or reducing the symptoms of corporal or gastrointestinal inflammation in an individual, comprising administering a composition comprising a bacteria of the genus Bacteroides, or a cellular constituent or derivative thereof according to claim 1.

11. A method for treating, delaying the onset of, or reducing the symptoms of cardiovascular disease in an individual, comprising administering a composition comprising a bacteria of the genus Bacteroides, or a cellular constituent or derivative thereof according to claim 1.

12. A method for treating, delaying the onset of, or reducing the symptoms of diabetes in an individual, comprising administering a composition comprising a bacteria of the genus Bacteroides, or a cellular constituent or derivative thereof according to claim 1.

13. A method for treating, delaying the onset of, or reducing the symptoms of colon cancer in an individual, comprising administering a composition comprising a bacteria of the genus Bacteroides, or a cellular constituent or derivative thereof according to claim 1.

14. A method for treating, delaying the onset of, or reducing the symptoms of gastrointestinal inflammation in an individual, comprising administering a composition comprising a bacteria of the genus Bacteroides, or a cellular constituent or derivative thereof according to claim 1.

15. A method according to claim 14, wherein the gastrointestinal inflammation is associated with a disease selected from the group consisting of Irritable Bowl Syndrome, Crohn's Disease, and colitis.

16. A method for treating, delaying the onset of, or reducing the symptoms of rheumatoid arthritis in an individual, comprising administering a composition comprising a bacteria of the genus Bacteroides, or a cellular constituent or derivative thereof according to claim 1.

17. A method for treating, delaying the onset of, or reducing the symptoms of asthma in an individual, comprising administering a composition comprising a bacteria of the genus Bacteroides, or a cellular constituent or derivative thereof according to claim 1.

18. A method for treating, delaying the onset of, or reducing the symptoms of multiple sclerosis in an individual, comprising administering a composition comprising a bacteria of the genus Bacteroides, or a cellular constituent according to claim 1.

19. A method according to claim 10, wherein the composition is administered in food or drink.

20. A genetically modified bacterium from the genus Bacteroides.

21. A composition for oral administration, comprising a genetically modified bacterium according to claim 20, and a physiologically acceptable carrier.

22. A synthetically derived molecule that is based upon a molecule/molecular pattern from a species within the genus Bacteroides.
